(19) United States
(12) Patent Application Publication
(10) Pub. No.: US 2010/0211090 A1
(43) Pub. Date: Aug. 19, 2010

(54) Medical Instrument For Cutting Tissue

(76) Inventor: Sascha Berberich, Tuttlingen (DE)

Correspondence Address: ST. ONGE STEWARD JOHNSTON & REENS, LLC, 986 BEDFORD STREET, STAMFORD, CT, 06905-5619, US

(21) Appl. No.: 12/706,577

(22) Filed: Feb. 16, 2010

(30) Foreign Application Priority Data
Feb. 16, 2009 (DE) 10 2009 010 561.1

(51) Int. Cl. A61B 17/32

(52) U.S. Cl. 606/170

(57) ABSTRACT

A medical instrument for cutting tissue has a tubular outer shaft having at least one outer shaft opening provided with at least one outer shaft blade. A tubular inner shaft is rotatable housed in the tubular outer shaft, the tubular inner shaft has at least one inner shaft opening provided with at least one inner shaft blade cooperating in a cutting action with said outer shaft blade. Said at least one inner shaft opening being curved and has a curved longitudinal central axis when viewed on a plane of projection running parallel to said rotation axis.

### BACKGROUND OF THE INVENTION

The invention relates to a medical instrument for cutting tissue.

From DE 10 2006 034 756 A1 a medical instrument for cutting tissue is known with a tubular outer shaft which, in the area of its distal end, has at least one outer shaft opening with at least one outer shaft blade, and with a tubular inner shaft which is rotatable about a rotation axis, is received in the outer shaft and, in the area of its distal end, has at least one inner shaft opening with at least one inner shaft blade which, when the inner shaft is moved in rotation, cooperates in a cutting action with the at least one outer shaft blade of the outer shaft.

Such instruments are used in minimally invasive surgery for detaching tissue in the human or animal body. To do so, the distal end of the shaft is guided to the operating site where the tissue that is to be detached is situated. To detach the tissue, the cutting element is moved in rotation by means of an external or internal drive having a motor. During the rotation, the blade formed on the cutting element cooperates in a cutting action with an edge of the outer shaft opening designed as a blade, by means of the blade of the cutting element passing the blade of the outer shaft opening upon each rotation. To ensure that the tissue to be detached is brought between the interacting blades, the shaft of such instruments is connected to a suction source, the suction effect of which reaches through the shaft as far as the outer shaft opening, in order to suck the tissue to be detached through this opening and into the shaft, such that the blades can detach the tissue. The detached tissue is sucked through the shaft by the partial vacuum.

The instrument known from DE 10 2006 034 756 A1 mentioned above comprises an outer shaft which, at its distal end, has a triangular or oval-shaped opening provided with a blade. A tubular rotatable inner shaft, at whose distal end a cutting element is formed, is received in the outer shaft. The cutting element has several oval-shaped openings which are likewise provided with a blade and, seen in a circumferential direction, are of different widths.

The tissue to be detached is sucked into one of the openings of the cutting element rotating in the outer shaft. The tissue is then detached by means of the blade-type edge of the cutting element opening, into which the tissue to be detached is sucked, running past the leading edge of the opening of the outer shaft in the direction of rotation. After the tissue has been detached, it is sucked through the inner shaft to the proximal end of the instrument.

Since the blade of the cutting element cooperating in a cutting action with the blade of the outer shaft has a convex profile, i.e. an outwardly curved profile, it is found that dense and tough tissue gathers centrally between the two blades and is actually cut off only at the end of a cutting phase, which often results in blockages. The reason for this is that, because of the unfavourable cutting angle that exists from the start of and during most of the cutting phase, this tissue is not in fact detached but instead “pressed” centrally between the two blades, and it is only broken up within a short time at the end of the cutting phase. The high cutting force required during the short cutting time leads to a marked increase in the cutting power that has to be applied at the end of the cutting phase, and this results in uneven loading of the cutting element and therefore adversely affects its smooth running.

Therefore, the disadvantage of the known instrument is that the tissue to be detached cannot be detached very efficiently, effectively and satisfactorily.

It is therefore an object of the present invention to develop an instrument of the type mentioned at the outset in such a way that the cutting performance or cutting action is improved.

### SUMMARY OF THE INVENTION

According to the invention, this object is achieved by a medical instrument for cutting tissue, comprising a tubular outer shaft having a distal end area, at least one outer shaft opening is provided in said distal end area of said tubular outer shaft, said at least one outer shaft opening having at least one outer shaft blade, a tubular inner shaft having a distal end area and a central longitudinal rotation axis, said tubular inner shaft being received in said tubular outer shaft and being rotatable in said tubular outer shaft about said central longitudinal rotation axis via a drive, at least one inner shaft opening is provided in said distal end area of said tubular inner shaft, said at least one inner shaft opening having at least one inner shaft blade, said at least one inner shaft blade, when said tubular inner shaft is rotated about said rotation axis cooperates in a cutting action with said at least one outer shaft blade, wherein said at least one inner shaft opening being curved and having a curved longitudinal central axis when viewed on a plane of projection running parallel to said rotation axis.

A considerable advantage of said curved inner shaft opening is that a sabre-like cutting action is afforded by the inner shaft opening having a curved longitudinal axis in a plane of projection running parallel to the rotation axis.

In other words, that is to say the inner shaft opening has a curved profile when seen in a side view. This profile has the effect that the inner shaft blades are likewise curved and sabre-shaped, as a result of which, in cooperation with the outer shaft blade, the effective cutting phase is prolonged and the cutting action is thus greatly improved. Accordingly, dense and tough fragments of tissue can be detached efficiently and continuously during a cutting procedure.

In another embodiment of the invention, the longitudinal axis of the at least one inner shaft opening runs along of an arc of a circle.

This measure has the advantage of resulting in a steady and continuous cutting profile, as a result of which the cutting performance and cutting action are improved and the smooth running of the rotating inner shaft is enhanced.

In another embodiment of the invention, said distal end area of said tubular inner shaft is being closed by a cap having a distal end point, said at least one inner shaft opening extends into said cap.

This measure further increases the flexibility to the extent that tissue located in the area of the distal end of the outer shaft, and therefore accessible only with difficulty, can also be sucked in and detached.

In another embodiment of the invention, the at least one inner shaft opening extends next to said end point.

If there are several inner shaft openings, this measure has the effect that the inner shaft openings do not intersect one another and also do not run to the same point, and the stability and strength of the inner shaft are thereby increased.

In another embodiment of the invention, the at least one inner shaft blade of the at least one inner shaft opening has a wedge-shaped cross section with a wedge point that is arranged on an outer face of the inner shaft.

This measure has the advantage that the cutting characteristics and the cutting action of the instrument according to the invention are further improved by the inner shaft blade tapering to a point. Moreover, depending on the area of use and on the tissue consistency, it is possible to use an instrument with a suitable cutting angle for achieving the maximum cutting efficiency.

With the wedge points arranged on the outer face of the inner shaft and the inner face of the outer shaft, the sharp wedge edges of both blades can work close together in a cutting action.

In another embodiment of the invention, the at least one outer shaft blade of the at least one outer shaft opening has a wedge-shaped cross section with a wedge point that is arranged on an inner face of the outer shaft.

This measure has the advantage that the cutting characteristics and the cutting action of the instrument according to the invention are further improved by the outer shaft blade tapering to a point. Moreover, depending on the area of use and on the tissue consistency, it is possible to use an instrument with a suitable cutting angle for achieving the maximum cutting efficiency.

In another embodiment of the invention, the inner shaft has three circumferentially offset inner shaft openings.

This measure has the advantage that several cutting procedures can take place during a complete rotation of the inner shaft. This results in a very high degree of efficiency of the cutting performance and the cutting action. Moreover, depending on the area of use and on the tissue consistency, i.e. depending on the required aggressiveness of the cutting, an instrument with a suitable cutting angle can be used in order to achieve the maximum cutting efficiency.

In another embodiment of the invention, the width of the inner shaft openings, seen in a circumferential direction, is smaller than the distance between the inner shaft openings.

This measure contributes further to the stability and strength of the inner shaft at the distal end.

In another embodiment of the invention, the outer shaft has three circumferentially offset outer shaft openings.

This measure has the advantage that the cutting performance of the instrument according to the invention can be further improved. An outer shaft with three outer shaft openings can be combined with an inner shaft with three inner shaft openings in order to enhance still further the cutting performance of the instrument according to the invention. This has the effect that many more blades are used and, as a result, more cutting procedures are performed, which also means that more tissue can be detached within a short time.

In another embodiment of the invention, the inner shaft can be moved in both directions of rotation by means of a motor of the drive.

Since different pairs of blades cooperate according to the direction of rotation, the cutting profile also changes, as also does the aggressiveness of cutting of the instrument. This measure therefore has the advantage that, depending on the area of use and on the tissue consistency, i.e. depending on the required aggressiveness of the cutting, a suitable direction of rotation can be chosen in order to achieve the maximum cutting efficiency.

In another embodiment of the invention, the inner shaft can be moved in oscillation over a predefined angle range.

This measure has the advantage that, on the one hand, two different cutting profiles with different cutting aggressiveness can be carried out in succession, and, on the other hand, the danger of blockage of the inner shaft is further reduced, since the risk of tissue fragments adhering is minimized by the “vibrating” movement. Moreover, in such an embodiment of the instrument according to the invention, the blades work even more efficiently, as a result of which more tissue can be detached more quickly.

In another embodiment of the invention, a range of said oscillation is defined in that one cutting procedure is carried out upon each excursion of the oscillating inner shaft.

This measure has the advantage that the excursion is minimized and the number of cutting procedures carried out at a time is maximized.

It will be appreciated that the aforementioned features and those still to be explained below can be used not only in the cited combinations, but also in other combinations, without departing from the scope of the present invention.

### BRIEF DESCRIPTION OF THE DRAWINGS

The invention is described and explained in more detail below on the basis of a chosen illustrative embodiment and with reference to the drawings, in which:

FIG. 1 shows a side view, in partial cross section, of a medical instrument for cutting tissue,

FIG. 2 shows an enlarged view of the distal end portion of the instrument from FIG. 1,

FIG. 3 shows a perspective view of the distal end portion of the inner shaft with three inner shaft openings,

FIG. 4 shows a cross section along the line A-A in FIG. 2,

FIGS. 5A-5C show a cutting procedure with three different positions of the inner shaft opening according to the illustrative embodiment from FIG. 1 and FIG. 2, and

FIGS. 6A-6C show a cutting procedure with three different positions of the inner shaft opening according to a further illustrative embodiment.

### DETAILED DESCRIPTION OF PREFERRED EMBODIMENTS

A medical instrument shown in the figures, and used for cutting tissue, is designated in its entirety by reference number 10.

The medical instrument 10 comprises a tubular outer shaft 12 which, at its proximal end, is connected to a housing 16.

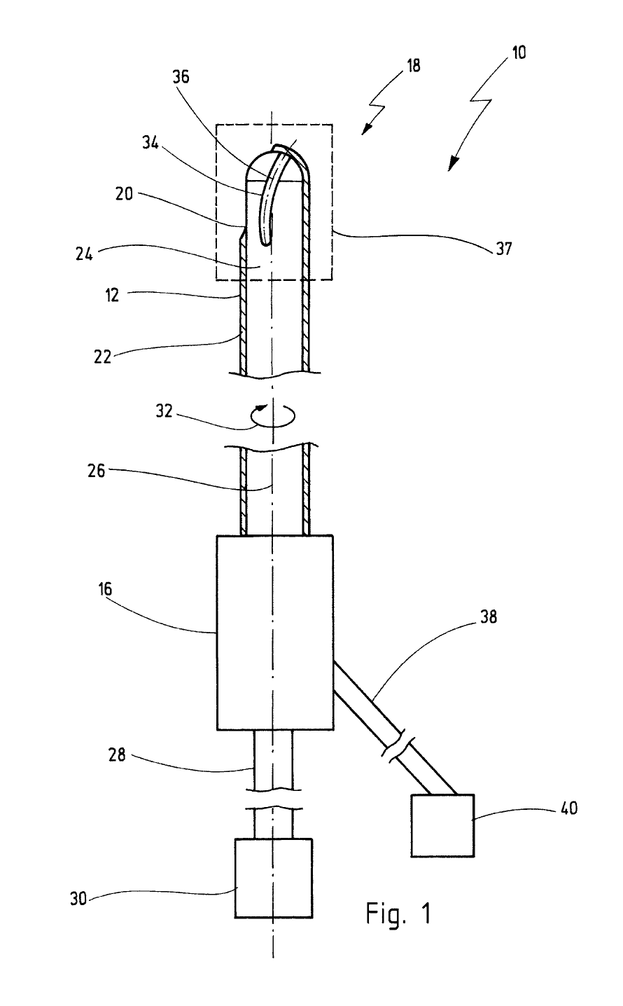

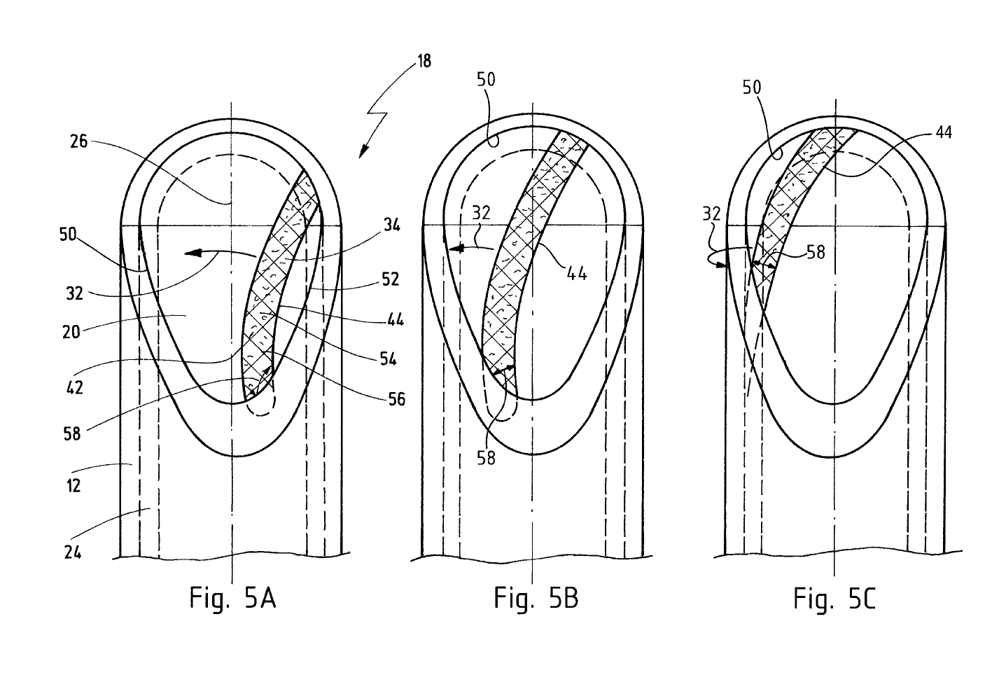

In its rounded and closed distal end 18, the outer shaft 12 comprises one outer shaft opening 20. The outer shaft opening 20 is formed by a circumferentially and axially limited opening of approximately oval shape being made in a wall 22 of the outer shaft 12, as can be seen from FIG. 1 in conjunction with FIG. 5.

A tubular inner shaft 24 is received in the outer shaft 12, in such a way as to be able to rotate therein about a central longitudinal rotation axis 26, and is connected at the proximal end to a motor 30 of a drive via a drive shaft 28. The drive shaft 28 is moved in rotation by the motor 30 according to the rotation arrow 32, and the rotary movement of the drive shaft 28 is transmitted to the inner shaft 24, which is moved in rotation relative to the stationary outer shaft 12.

An outer diameter of the tubular inner shaft 24 corresponds approximately to an inner diameter of the tubular outer shaft 12.

It will further be noted that the motor 30 can also be configured such that the inner shaft 24 can additionally be moved in the direction counter to the rotation arrow 32 or in oscillation in both directions.

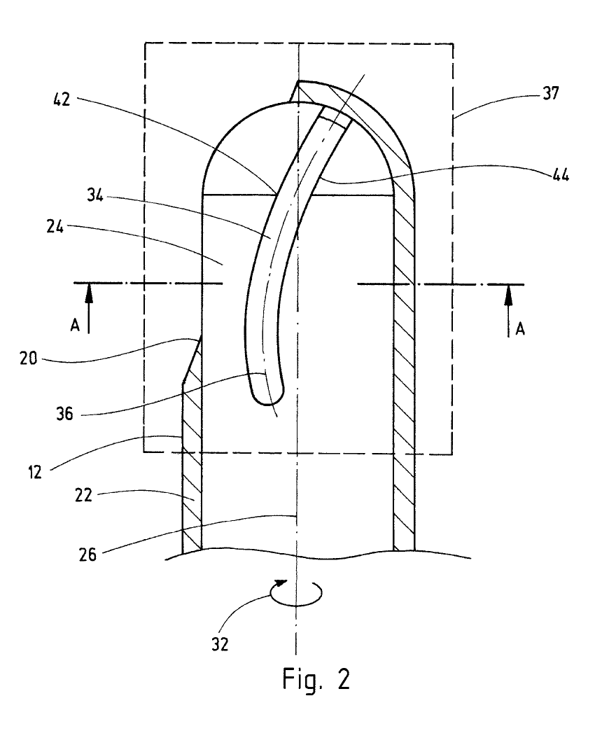

At the distal end 18, in the area of the outer shaft opening 20 of the outer shaft 12, the inner shaft 24 has an inner shaft opening 34. The inner shaft opening 34 extends along a curved longitudinal axis 36 and has an elongate sabre shape, as can be seen in detail from FIG. 2. The curved course of the longitudinal axis 36 results from projecting the longitudinal axis 36 onto a plane of projection 37 running parallel to the rotation axis 26.

Moreover, the instrument 10 is connected to a suction source 40 via a suction nozzle 38 of the housing 16. With the suction source 40 switched on, a suction stream forms through the inner shaft 24 to as far as the inner shaft opening 34, which stream is directed from the inner shaft opening 34 to the suction nozzle 38.

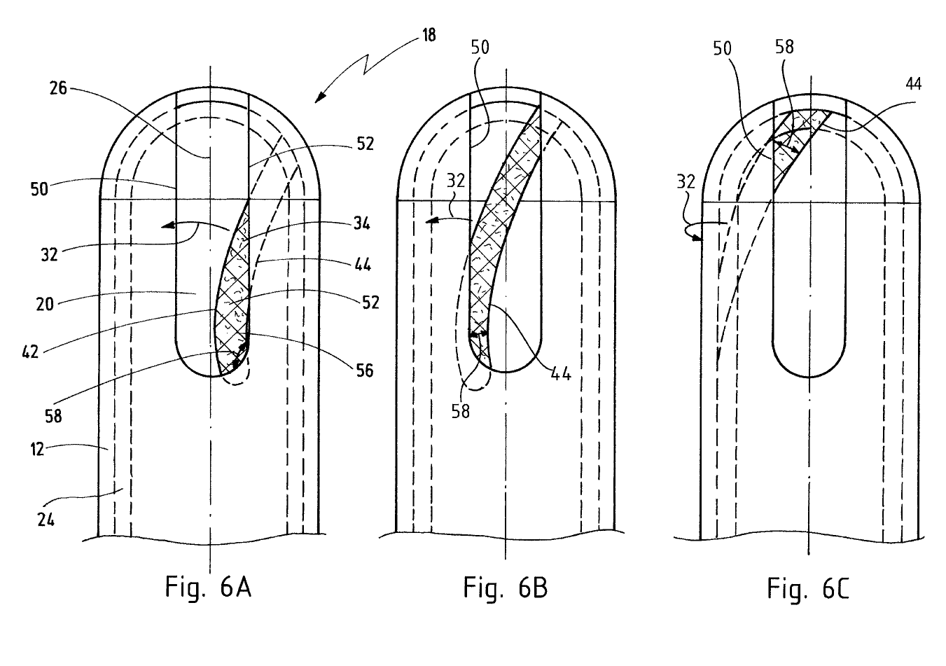

As can be seen from the enlarged view in FIG. 2, the inner shaft 24 has a first inner shaft blade 42, which leads in the direction of rotation, and a second inner shaft blade 44, which trails in the direction of rotation. In the illustrative embodiment shown, the second inner shaft edge 44 cooperates in a cutting action with the outer shaft 12, as will be described in detail below in the description of FIGS. 5 and 6.

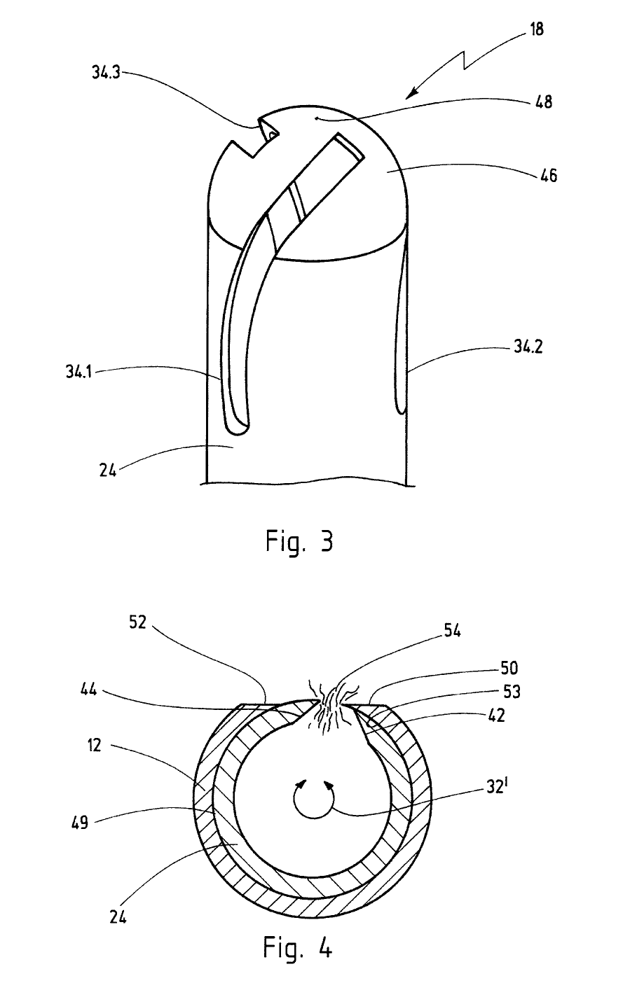

FIG. 3 is a perspective view of the distal end 18 of the inner shaft 24 from FIG. 1 and FIG. 2.

The inner shaft 24 has three circumferentially offset inner shaft openings 34.1, 34.2, 34.3. The inner shaft openings 34.1, 34.2, 34.3 extend as far as a distal end face 46 shaped as a cap, specifically next to a distal end point 48 of the cap. The end point 48 forms the apex of the distal end 18, such that they neither intersect each other nor run to the distal end point 48. As has already been described above, this design leads to increased stability and increased strength of the inner shaft 24 at the distal end 18.

It will also he noted that the outer shaft 12, which cooperates with an inner shaft 24 of this kind, can also have three circumferentially offset outer shaft openings 12.1, 12.2, 12.3, in order to further increase the cutting performance.

In the sectional view shown in FIG. 4, along section line A-A from FIG. 2, the first and second inner shaft blades 42, 44 of the inner shaft opening 34 each have a wedge-shaped cross section with a wedge point arranged close to an outer face 49 of the inner shaft 24, in order to increase the aggressiveness of the cutting. Moreover, the outer shaft opening 20 has a first outer shaft blade 50 and a second outer shaft blade 52, which are likewise wedge-shaped and each have a wedge point arranged at the level of the inner face 53 of the outer shaft 12. The aggressiveness of the cutting can be increased still further by this means.

A rotation arrow 32′ indicates the possibility that the inner shaft 24 can move in both directions of rotation, such that a cutting procedure for cutting a tissue 54 can be performed on both outer shaft blades 50, 52. In the illustrative embodiment shown, the inner shaft 24 rotates in the clockwise direction, such that the second inner shaft blade 44 cooperates in a cutting action with the first outer shaft blade 50 and the tissue 54 can be efficiently detached.

Upon rotation in the opposite direction, e.g. during an oscillating movement, the first inner shaft blade 42 and the second outer shaft blade 52 cooperate analogously in a cutting action and generate the corresponding cutting procedure.

The use of the medical instrument 10 from FIG. 1 will be explained in brief on the basis of the sequence of FIGS. 5A-C and 6A-C.

FIG. 5 and FIG. 6 illustrate the advantageous cutting profile and the improved cutting action of the instrument according to the invention on the basis of the curved, sabre-shaped inner shaft blade 44. Three positions A, B, C of the inner shaft 24 during the passage of the inner shaft opening 34 through the outer shaft opening 20 are shown in each case. The outer shaft opening 20 is oval-shaped (FIG. 5) or elongate (FIG. 6). As soon as the inner shaft opening 34 has passed the outer shaft opening 20, a suction area 56 forms, which is indicated as a cross-hatched area, and the tissue 54 is sucked into this area by the suction current generated by the suction source 40. By means of the suction effect present in the suction area 56, the tissue 54 to be detached is sucked in through the outer shaft opening 20 and the inner shaft opening 34. As the second inner shaft blade 44 of the inner shaft 24 runs past the first outer shaft blade 50 of the outer shaft 12, as can be seen from FIGS. 5 and 6, the tissue 54 that is to be detached, and that has been sucked in, is cut off. The detached tissue 54 is sucked through the inner shaft 24 to the proximal end of the instrument 10.

The cutting angle 58 arising during a cutting procedure between the rear, and therefore cutting, second inner shaft blade 44 and the first outer shaft blade 50 cooperating therewith varies throughout the movement of the inner shaft opening 34 past the outer shaft opening 12.

FIG. 5A and FIG. 6A show the “suction phase” during which tissue 54 is only sucked in, since there is virtually no cutting action because of the obtuse cutting angle 58. As is evident from FIG. 5B and FIG. 6B, the “cutting phase” of the instrument according to the invention, compared to the prior art described at the outset, begins at a very early stage and with an advantageously acute cutting angle 58. The curved second inner shaft blade 44 thus cooperates very effectively with the first outer shaft blade 50 and in so doing cuts through the tissue 54 like a sabre. Finally, FIG. 5C and FIG. 6C show the “end phase” in which it is clear that the acute cutting angle slowly and continuously shrinks during the passage, thus imitating the cutting profile of slowly closing shears, for example anvil shears, and providing a more efficient cutting action during a longer cutting phase in relation to the tissue 54 that is to be removed.

1. A medical instrument for cutting tissue, comprising
a tubular outer shaft having a distal end area,
at least one outer shaft opening is provided in said distal end area of said tubular outer shaft, said at least one outer shaft opening having at least one outer shaft blade,
a tubular inner shaft having a distal end area and a central longitudinal rotation axis, said tubular inner shaft being received in said tubular outer shaft and being rotatable in said tubular outer shaft about said central longitudinal rotation axis via a drive,
at least one inner shaft opening is provided in said distal end area of said tubular inner shaft, said at least one inner shaft opening having at least one inner shaft blade,
said at least one inner shaft blade, when said tubular inner shaft is rotated about said rotation axis cooperates in a cutting action with said at least one outer shaft blade,
wherein
said at least one inner shaft opening being curved and having a curved longitudinal central axis when viewed on a plane of projection running parallel to said rotation axis.

2. The medical instrument of claim 1, wherein said curved longitudinal central axis runs along an arc of a circle.

3. The medical instrument of claim 1, wherein said distal end area of said tubular inner shaft being closed by a cap having a distal end point, said at least one inner shaft opening extends into said cap.

4. The medical instrument of claim 3, wherein said at least one inner shaft opening extends next to said end point.

5. The medical instrument of claim 1, wherein said at least one inner shaft blade of said at least one inner shaft opening has a wedge-shaped cross section having a wedge point arranged on an outer face of said inner shaft.

6. The medical instrument of claim 1, wherein said at least one outer shaft blade of said at least one outer shaft opening has a wedge-shaped cross section having a wedge point arranged on an inner face of said outer shaft.

7. The medical instrument of claim 1, wherein three inner shaft openings are provided in said inner shaft, said three inner shaft openings are circumferentially offset one to another.

8. The medical instrument of claim 7, wherein a width of said three inner shaft openings, seen in a circumferential direction, being smaller than a distance between two neighboured inner shaft openings.

9. The medical instrument of claim 7, wherein said outer shaft has three outer shaft openings being circumferentially offset one to another.

10. The medical instrument of claim 1, wherein said inner shaft can be rotated by said drive in two opposite directions of rotation.

11. The medical instrument of claim 10, wherein said inner shaft can be moved in oscillation over a predefined angle range.

12. The medical instrument of claim 11, wherein said angle range of said oscillation being defined in that one cutting procedure is carried out upon each excursion of oscillation of said inner shaft.
